(19) United States
(12) Patent Application Publication
(10) Pub. No.: US 2020/0129583 A1
(43) Pub. Date: Apr. 30, 2020

(54) COMPOSITION FOR TREATING ACUTE URINARY RETENTION

(71) Applicant: ANTEV LIMITED, London (GB)

(72) Inventor: Finn LARSEN, Hawick (GB)

(21) Appl. No.: 16/624,900

(22) PCT Filed: Jun. 28, 2018
(86) PCT No.: PCT/EP2018/067425

§ 371 Date: Dec. 19, 2019

(51) Int. Cl. A61K 38/09; A61K 45/06; A61P 13/08; A61K 9/10; A61K 31/519; A61K 31/4985; A61K 31/53; A61K 31/506; A61K 31/473; A61K 31/565

(52) U.S. Cl. CPC A61K 38/09; A61K 45/06; A61P 13/08; A61K 9/10; A61K 31/565; A61K 31/4985; A61K 31/53; A61K 31/506; A61K 31/473; A61K 31/519

(57) ABSTRACT

The present invention provides a composition for treating acute urinary retention and reducing the recurrences rates in a male patient by administering a composition for treating acute urinary retention, said composition comprises at least one GnRH antagonist and at least one alpha blocker and/or at least one 5-alpha reductase inhibitor. Said composition provides a rapid reduction in the patient's testosterone level and returns the patient's testosterone level to near baseline in less than eight weeks. Also, the administration of the GnRH antagonist provides a significant reduction in the patient's acute urinary retention symptoms for a medication period of at least four months, six months or more.

Acute Urinary Retention (AUR) is a condition characterized by a sudden inability to urinate that is invariably but not always painful; painless AUR is rare and is usually associated with CNS pathology. Over 10% of men in their seventh decade will experience AUR over a 5-year period (Jacobsen S J, Jacobsen D J, Girman C J et al. Natural history of prostatism: risk factors for acute urinary retention. J Urol 1997; 158: 481-7) and the risk increases to one in three over 10 years.

Acute urinary retention (AUR) is one of the most significant complications or long-term outcomes of benign prostatic hyperplasia (BPH).

BPH may be treated by surgery to remove prostatic tissue. This reduces the physical bulk of the prostate, thereby reducing obstruction and urinary symptoms. Transurethral resection of the prostate (TURP) is the golden standard surgical treatment for urinary symptoms due to BPH. The procedure is effective and tolerated reasonably well, although associated with postoperative morbidity like urinary incontinence and retrograde ejaculation. As a consequence, although TURP is effective, it is considered as a last resort, and medicinal therapy has become the first line treatment of symptomatic BPH.

Between 25% and 30% of men who underwent transurethral resection of the prostate (TURP) had AUR as their main indication (Holtgrewe H L, Mebust W K, Dowd J B, et al. Transurethral prostatectomies: practical aspects of the dominant operation in American urology. J Urol. 1989; 141:248-253). Today most patients failing to urinate after an attempt of catheter removal still undergo surgery.

From an economic point of view, AUR is a significant event, and from the patient's point of the, often a feared one. For the patient it presents as the inability to urinate, with increasing pain, a visit to the emergency room, catheterization, follow-up visits to the physician, an attempt to remove the catheter, and eventually recovery or surgery, all in all a painful and time-consuming process. In the literature, the risk of recurrent AUR has been cited as being 56%-64% within week of the first episode and 76%-83% in men with diagnosed BPH. (Klarskov P, Andersen J T, Asmussen C F, et al. Symptoms and signs predictive of the voiding pattern after acute urinary retention in men. Scand J Urol Nephrol. 1987; 21:23-28).

The primary treatment goals for men with AUR are to decompress the urinary bladder, alleviate lower urinary tract symptoms and prevent the progression of the disease, in particular the need for surgery. The risk of progression is directly related to the prostate volume. Therefore, drugs that reduce prostate volume have been shown to exhibit the greatest effect in preventing the disease progression. Two classes of drugs, α-blockers and 5-α-reductase inhibitors, are presently approved by authorities for treatment of symptoms of BPH. It is believed that al receptors influence lower urinary tract function not only through the direct effect on smooth muscle but also at the level of the spinal cord, ganglia, and nerve terminals to influence sympathetic, parasympathetic, and somatic outflows to the bladder, bladder neck, prostate, and external urethral sphincter. The 5-α-reductase inhibitors have been shown to reduce prostate volume and reduce the risks of acute urinary retention and need for BPH surgery.

Although the currently available drugs may significantly improve lower urinary tract symptoms, there is a substantial number of patients who do not benefit from this treatment. The magnitude of improvement with current medicinal therapy (as measured by International prostate symptom score (I-PSS)) does not compare to the results achieved with surgical treatments. There is therefore a need for an improved medicinal therapy, which is well tolerated and comparable or more comparable with surgery in terms of efficacy and may even delay the need for surgical intervention.

Accordingly, the need for a composition for treating acute urinary retention (AUR) with an improved safety profile and/or improved patient compliance exists.

This need is met according to the present invention, which provides a composition for treating acute urinary retention, said composition comprises at least one GnRH antagonist and at least one α-blocker and/or at least one 5α-reductase inhibitor.

The combination of at least one GnRH antagonist and at least one α-blocker and/or at least one 5α-reductase inhibitor provides an unforeseen synergistic effect by rapidly providing a significant reduction in the patient's acute urinary retention symptoms and the size of the prostate.

Since testosterone is responsible for a number of male characteristics, reduced and/or low testosterone levels provides a number of physiological changes, such as diminished interest in sex, impotence, reduced lean body mass, decreased bone density, lowered mood, and energy levels. The at least one GnRH antagonist will provide a reduction in the patients testosterone level, however since the testosterone level will not remain low but return to the baseline levels in less than eight weeks, these side effects are substantially prevented by using the composition according to the present invention, even though the positive effects on the patients AUR is maintained for a complete medication period of at least four to six months. Thus, by using the composition according to the invention the problems of long-term low testosterone levels in a male patient is effectively prevented.

In contrast to the treatments known in the art where the patients are to have daily or weekly administrations, the present composition only requires administration of a single or two dosages. In the latter case, the two dosages are separated by an interval of about 48 hours. Accordingly, the very few administrations increases patient acceptance and compliance of therapy.

The composition according to the invention is unique since one or two dosages of said composition is sufficient for relieving the patient of the AUR symptoms in a medication period of about six month, and at the same time preventing problems of long-term low testosterone levels in a male patient.

In a preferred embodiment according to the invention the GnRH antagonist is N-Ac-d-Nal¹, d-pCl-Phe², d-Pal³, d-(Hci)⁶, Lys(iPr)⁸, d-Ala¹⁰ trifluoroacetate (Teverelix TFA), however other GnRH antagonist, such as abarelix, cetrorelix, degarelix, ganirelix, ozarelix, antide, or a salt of any of these are also contemplated within the scope of the present invention. In the present application the term “GnRH antagonist” should be construed to comprise a salt thereof even if not explicitly recited.

The composition according to the invention preferably comprises from about 30 mg to about 90 mg of the GnRH antagonist, preferably between 45 and 80 mg, and even more preferred about 60 mg, as the inventors has found that said amount of GnRH antagonist is sufficient for providing the desired response in the patient, i.e. a reduction to 50% of the patient's baseline testosterone level is obtained immediately after administration of the composition, and wherein the testosterone level in the patient returns to near baseline in less than eight weeks.

It is further preferred that the GnRH antagonist is administered in a sustained release formulation, allowing a more uniform and optimal plasma drug profile, and a smoother therapeutic response over the dosage interval. Clinically, this offers the potential to optimise drug therapy and decrease the occurrence of concentration-related adverse effects, reduce the exposure to drug and reduce the cost of drug therapy. In addition, sustained release formulations may increase patient acceptance and compliance of therapy.

The sustained release composition can in a preferred embodiment be in the form of a microcrystalline aqueous suspension, preferably those disclosed in WO 2003/022243. Said document discloses a method in which the GnRH antagonist, (which is a hydrophobic peptide) is contacted with a counter-ion in an amount and at a molar ratio sufficient to provide a fluid, milky microcrystalline aqueous suspension of the GnRH antagonist without formation of a gel.

Thus, the composition according to the invention has both an immediate onset of action and a sustain release of antagonist thereby ensuring that the subject maintains a therapeutically effective concentration in the blood plasma. This will not only provide a more reliable composition for the treatment of AUR, but also improve patient compliances as fewer administrations (e.g. injections) are required.

Accordingly, the composition is advantageously not only in reducing the symptoms associated with AUR but also in reducing recurrences rates in a patient for a prolonged period of time of at least four to six months, and at the same time prevent side-effects of reduced levels of testosterone. The composition according to the invention may even delay or prevent the need for surgical intervention.

Since the composition comprises at least one GnRH antagonist and at least one α-blocker and/or at least one 5α-reductase inhibitor, the inventors have found that the patients AUR symptoms can be relieved even faster than the individual components administered alone.

Normally prostate development occurs under the influence of dihydrotestosterone (DHT), which is a derivative of testosterone with a higher affinity for the androgen receptor. The conversion of testosterone to dihydrotestosterone occurs by the enzyme 5α-reductase; and DHT production can therefore be inhibited by 5α-reductase inhibitors causing involution of the prostatic epithelium and slowing the progression of AUR.

Even though it is known that 5α-reductase inhibitors reduce prostate volume and thereby reduce the obstructive component associated with AUR, 5α-reductase inhibitors have to be administered on a daily basis to ensure that the testosterone is not converted into dihydrotestosterone. However, due to the undesirable side effects of 5α-reductase inhibitors, e.g. dizziness and impotence, a daily treatment is obviously not desirable. However, by administering the 5α-reductase inhibitor in the same composition as (or together with) an GnRH antagonist e.g. teverelix TFA, the testosterone concentration will not only be reduced; but the testosterone already present in the patient will effectively be prevented from being converted into dihydrotestosterone. Thus, the patient will experience an onset of action which is significantly faster by using the composition according to the invention compared to the individual components given alone. Accordingly, the patient's symptoms are relieved almost instantly when the composition comprises both a GnRH antagonist and a 5α-reductase inhibitor. Furthermore, since the patient is only exposed to extremely small dosages of the at least one 5α-reductase inhibitor and only for a limited duration, compared to the conventional dosage regimes where the 5α-reductase inhibitor is administered daily, the patients will not experience the severe side effects normally associated with 5α-reductase inhibitor

The 5α-reductase inhibitor may be any suitable 5α-reductase inhibitor, it is however preferred that said 5α-reductase inhibitor is selected from the group comprising dutasteride, finasteride, episteride, and alfatradiol.

Depending on the 5α-reductase inhibitor used, the composition according to the invention may comprise from about 0.5 mg to about 5 mg of the 5α-reductase inhibitor. As an example can be mentioned that if the 5α-reductase inhibitor is dutasteride the composition according to the invention may preferably comprise about 0.5 mg, and if the 5α-reductase inhibitor is finasteride the composition according to the invention may preferably comprise about 5 mg, however said dosage may be varied depending on the used 5α-reductase inhibitor.

AUR may also be at least partially caused by increased sympathetic activity at the level of the prostatic smooth muscles, and it is known that α-blockers (alpha-1 adrenoreceptor antagonists) will reduce the symptoms associated with AUR in this respect by relaxing the smooth muscles of the prostate and bladder neck and thereby improve urine flow. However, α-blockers are also associated with adverse effects such as orthostatic hypotension, dizziness, and sexual dysfunction (ED, abnormal ejaculation), and the α-blockers alone will not provide the desired effects.

However, the inventors of the present invention have found that by combining a GnRH antagonist with at least one α-blocker the patient will not only experience a fast onset of action but also a prolonged duration of action before an additional dosage has to be administered. Similar to the 5α-reductase inhibitor, the patient is only exposed to extremely small dosages of the at least one α-blocker and only for a limited duration, thus the patients will not experience the severe side effects normally associated with the α-blockers.

The α-blocker may be any suitable α-blocker, it is however preferred that said α-blocker is selected from the group comprising phenoxybenzamine, phentolamine, tolazoline, trazodone, alfuzosin, doxazosin, prazosin, tamsulosin, terazosin, silodosin, carvedilol, and labetalol.

Depending on the α-blocker used, the composition according to the invention may comprises from about 1 mg to about 40 mg of the α-blocker. As an example can be mentioned that if the α-blocker is doxazosin or silodosin the composition according to the invention may preferably comprise about 8 mg, and if the α-blocker is prazosin the composition according to the invention may preferably comprise about 4 mg, however said dosage may be varied.

In a further embodiment of the composition according to the invention, the composition further comprises at least one phosphodiesterase type 5 inhibitor (PDE5-inhibitor), e.g. sildenafil, tadalafil, vardenafil, and avanafil. PDE5's are known to cause vasodilation in the penis, and studies using daily dosing of both sildenafil and tadalafil have shown improvement in International Prostate Symptom Score (IPSS) see e.g. McVary K T, Roehrborn C G, Kaminetsky J C, et al. Tadalafil relieves lower urinary tract symptoms secondary to benign prostatic hyperplasia. J Urol. 2007; 177:1401-1407. Thus, combining the composition according to the invention with at least one PDE5 may aid in reducing the symptoms associated with AUR.

In a preferred embodiment the composition according to the invention comprises least one GnRH antagonist, at least one α-blocker and at least one 5α-reductase inhibitor. Such a composition may e.g. be preferably in patients having severe AUR, as the combined treatment provides an improve response which ensures that the patients AUR symptoms can be relieved almost immediately after administration. A further benefit of adding both at least one α-blocker and at least one 5α-reductase inhibitor is that the amounts of the individual active ingredients can be reduced in the composition whereby the patient will be exposed to smaller amounts of α-blocker and 5α-reductase inhibitor, significantly reducing the side effects of said ingredients.

As examples of reduced symptoms of AUR obtained by using the composition according to the invention, can be mentioned a reduction in recurrence rates, an increased urinary flow and output, a decrease in residual urinary volume, a reduced prostate volume, reduced nocturia symptoms and a delay or elimination of the need for surgery.

The composition according to the invention is preferably administered as a single dosage (e.g. comprising 60 mg teverelix TFA) or two dosages (e.g. comprising 45 mg teverelix TFA), after which a medication period of at least four to six months is initiated, and wherein the administration of said composition provides about a 50% reduction in the patient's baseline testosterone level immediately after administration, and wherein the testosterone level in the patient returns to near baseline in less than eight weeks. If two dosages are administered, said two dosages is preferably separated by an interval of about 48 hours.

The dosage of GnRH antagonist preferably comprises about 30, 45, 60 mg or more of a GnRH antagonist, preferably Teverelix TFA, and about 0.5, 1 or 5 mg 5α-reductase inhibitor, and/or about 4, 8 or 10 mg α-blocker. In a preferred embodiment a signal dosage of the composition comprises about 60 mg Teverelix TFA, about 5 mg finasteride and/or about 5 mg doxazosin.

However, while the amount of GnRH antagonist, α-blocker and/or 5α-reductase inhibitor provided in the composition of the present invention may vary, the inventors have found that a single or two dosages of GnRH antagonist are sufficient for obtaining the reduction in symptoms of AUR in the patient for a medication period of at least four to six month, and preferably even longer.

Thus, the positive effects of the composition according to the invention on the patients AUR is maintained for a complete medication period of at least four to six months. In order to ensure a continued relive of the AUR symptoms and recurrence rate, it is preferred that an additional maintenance dosage of the composition according to the invention may be administered about six months after the prior administration. Thus, a patient in need of treatment only needs to administer one or two dosages of the composition according to the invention, e.g. at intervals of 48 hours, every six month, i.e. only twice a year. In an alternative embodiment the additional maintenance dosage administered about six month after the first or prior administration, is a composition comprising only one or more GnRH antagonist, e.g. teverelix TFA.

Using the composition according to the invention has shown, the patients testosterone level is reduced to at least 50% of the patient's baseline testosterone level shortly after administration of the GnRH antagonist. Within the context of the present invention the term “shortly” means within one to two days after the single dosage regime has been administered, but may in some situations be up to seven days after administration. If the dosage regime comprises two dosages administered within an interval of 48 h, the term refers “shortly” means that the testosterone level is reduced to 50% in one to two days (up to seven days) after administration of the second dosage.

The inventors of the present invention have surprisingly found that by administering the composition according to the invention, it is possible to obtain a rapid, but transient, reduction in the testosterone level in a male patient, thereby not only significantly reducing symptoms of AUR but also preventing problems of long-term low testosterone levels in a male patient.

Since body mass does not influence GnRH activity, the single dosage of GnRH antagonist is considered to be universal for all men. However, the exact dosage of the GnRH antagonist in the formulation will among others depend on the GnRH antagonist used, and/or if more than one GnRH antagonist is present in the composition.

Since the composition according to the present invention ensures that a patients AUR symptoms are reduced for a medication period of at least four to six month, and at the same time prevents the patient's server side effects of testosterone depletion, said composition provides a superior treatment composition compared to the known AUR treatments.

Not only is a very simple dosage regime provided but the treatment can be repeated every six months period, ensuring that the AUR symptom's are effectively reduced, recurrences rate reduced or even that the AUR is treated. Accordingly, the present invention provides a composition for effectively treating AUR and at the same prevents the risk of recurrence.

The composition according to the invention may be administered in a pharmaceutical formulation that is suitable prepared for the route of administration, which preferably is by injection.

The composition according to the invention may be administered as a single, combined composition, however in an alternative embodiment the individual active ingredients of said composition are administered separately in individual compositions, i.e. the GnRH antagonist is administered in one composition e.g. by injection, and the at least one α-blocker and/or at least one 5α-reductase inhibitor and/or the at least one phosphodiesterase type 5 inhibitor, are each administered in their separate composition, e.g. orally. In embodiments were the active ingredients are administered in separate composition it is however preferred that the respective active ingredients are administer substantially simultaneously, i.e. as simultaneously as practically possible for the person administering said ingredients. In any case it is preferred that the respective active ingredients are administered within one hour and not more than two hours.

Even though it is preferred to administer the composition according to the invention as a single or two dosages, it is possible to separately continue the administration of the at least one α-blocker and/or the at least one 5α-reductase inhibitor during a treatment period, e.g. the entire treatment period, if desired.

The GnRH antagonist can be administered in a sustained release formulation, preferably in a microcrystalline aqueous suspension. The dosage of GnRH antagonist preferably comprises between about 30 and 90 mg, e.g. 30, 45, 60 mg of a GnRH antagonist, preferably Teverelix TFA.

The invention also relates to the use of a pharmaceutical formulation comprising a therapeutically effective amount of a GnRH antagonist to rapidly reduce a patient's testosterone level and to return the patient's testosterone level to near baseline in less than eight weeks, wherein the pharmaceutical formulation is administered as a single dosage regimen of the amount of GnRH antagonist and the amount of GnRH antagonist provides a significant reduction in the patient's acute urinary retention symptoms for a medication period of at least four months, six months or more.

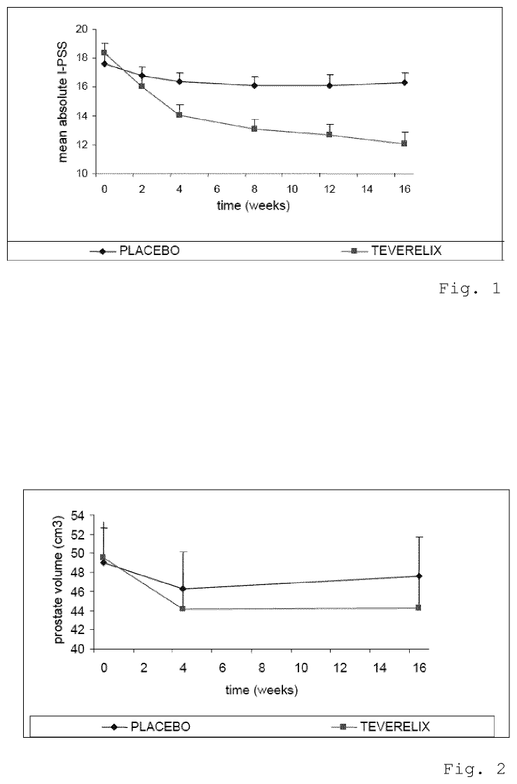

FIG. 1 is a graph showing the mean I-PSS score of patients treated with Teverelix TFA,

FIG. 2 is a graph showing the mean prostate volume of patients treated with Teverelix TFA,

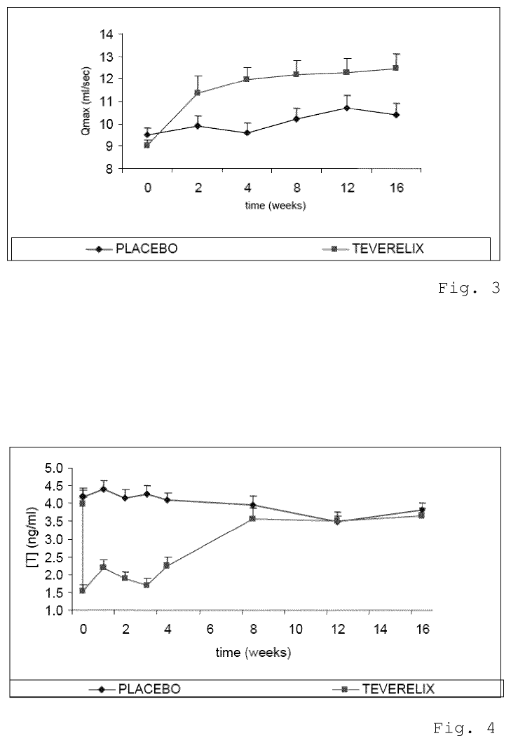

FIG. 3 is a graph showing the mean urinary flow rate of patients treated with Teverelix TFA,

FIG. 4 is a graph showing the mean testosterone level in serum of subjects treated with Teverelix TFA, using the composition according to the invention.

It has surprisingly been found that by administering the composition according to the invention as a single or two dosages, it is possible to obtain a rapid, but transient, reduction in the testosterone level in a male patient, and thereby not only significantly reducing symptoms of LUTS, and AUR including a reduction in recurrence rates, but also preventing problems of the longer-term effects of low testosterone levels in a male patient.

### EXAMPLES

A number of examples were conducted in order to substantiate that patients can obtain a significant reduction in AUR symptoms.

### Example 1

A phase II randomised, double blind, placebo-controlled, multi-centre, multi national study was conducted in order to investigate the use of an Teverelix TFA formulation in the composition according to the invention. In said study the International Prostate Symptom Score (I-PSS), the prostate volume, the urinary flow rate and the testosterone level in males suffering from BPH were evaluated.

The Teverelix TFA formulation was reconstituted as follows. 0.8 ml 5% mannitol was added to 60 mg of the LHRH antagonist Teverelix trifluoroacetate. The mixture was stirred using vortex during one minute providing a dosage formulation of Teverelix TFA as a flowing milky pearly microcrystalline aqueous suspension. The suspension is made of microcrystals of about 10 μm length. Corresponding placebo formulations, without Teverelix TFA, were also prepared

Formulation A: 60 mg Teverelix TFA in 0.8 ml 5% mannitol

Formulation A-placebo: 0.8 ml 5% mannitol

Two single injections of Formulation A and Formulation A-placebo was administered s.c. at an interval of 48 hours to 81 male patients (41 on A and 40 on A-placebo) suffering from BPH.

International Prostate Symptom Score

The International Prostate Symptom Score (I-PSS) is based on the answers to seven questions concerning urinary symptoms and one question concerning quality of life. Each question concerning urinary symptoms allows the patient to choose one out of six answers indicating increasing severity of the particular symptom. The answers are assigned points from 0 to 5. The total score can therefore range from 0 to 35 (asymptomatic to very symptomatic).

The results of the male patients I-PSS answers are summarized in FIG. 1, and it is clear that the patients in the Teverelix TFA group showed an 39.9% improvement on I-PSS compared to 7.4% in the placebo group. The symptoms improved in the Teverelix group within two weeks and continued to improve over the entire 16-week duration of the study.

Prostate Volume

The result relating to the protate volume is shown in FIG. 2 and it is evident that the prostate volume decreased by 11.5% in the Teverelix TFA group. This reduction occurred within four weeks of initiating therapy. By reducing the physical bulk of the prostate, the obstruction and urinary symptoms were reduced.

Urinary Flow Rate

The results relating to the urinary flow rate are depicted in FIG. 4, showing that in the Teverelix TFA group there was a 43% increase in maximum urinary flow rate (Qmax). A significant increase in the flow rate can be seen within the first two weeks. Most likely this effect begins to occur almost immediately.

Testosterone Level

As is shown in FIG. 4, the immediate reduction in the testosterone level in the Teverelix TFA group is responsible for the prompt relief of symptoms. Since the testosterone level returns to baseline levels within eight weeks, the composition will not only significantly reduce symptoms of AUR but also preventing problems, such as impotence and reduced lean body mass, normally associated with low testosterone levels in a male patient.

In summary, Teverelix TFA competitively binds with endogenous GnRH at hypothalamic receptors results in a dose-dependent reduction in testosterone secretion and subsequently a reduction in the size of the prostate.

In particular:
Qmax: increases by 43% at 16 weeks
IPSS: exhibits a 12.6% reduction at 2 weeks; 33.9% reduction at 16 weeks
QoL: exhibits a 32.8% improvement in quality of life score at 16 weeks

In addition, no serious side effects have been reported with the use of Teverelix TFA

In accordance with the present invention, a regimen or dose of GnRH antagonist is provided which is effective to reduce AUR but is ineffective to substantially reduce production of testosterone for more than eight weeks.

The formulations used in the present invention is inexpensive to manufacture, and due to the ease of use it provides a very simple dosage regime.

Modifications and combinations of the above principles and combinations are foreseen within the scope of the present invention.

1. A composition for treating acute urinary retention, said composition comprises at least one GnRH antagonist and at least one alpha blocker and/or at least one 5-alpha reductase inhibitor.

2. A composition according to claim 1, wherein the GnRH antagonist is selected from the group comprising abarelix, cetrorelix, degarelix, ganirelix, ozarelix, antide, teverelix, and a salt of any of the above.

3. A composition according to claim 1, wherein the GnRH antagonist is N-Ac-d-Nal1,d-pCl-Phe2,d-Pal3,d-(Hci)6,Lys(iPr)8,d-Ala10 trifluoroacetate (Teverelix TFA).

4. A composition according to claim 1, wherein the GnRH antagonist in the composition is in the form of a microcrystalline aqueous suspension.

5. A composition according to claim 1, wherein the composition comprises from about 30 mg to about 90 mg of the GnRH antagonist, preferably between 45 and 80 mg, and even more preferred about 60 mg.

6. A composition according to claim 1, wherein the alpha blocker is selected from the group comprising phenoxybenzamine, phentolamine, tolazoline, trazodone, alfuzosin, doxazosin, prazosin, tamterazosin, silodosin, carvedilol, and labetalol.

7. A composition according to claim 1, wherein the composition comprises from about 0.5 mg to about 5 mg of the alpha blocker, preferably between 1 and 4, and even more preferred about 2 mg.

8. A composition according to claim 1, wherein the 5α-reductase inhibitor is selected from the group comprising dutasteride, finasteride, episteride, and alfatradiol.

9. A composition according to claim 1, wherein the composition comprises from about 1 mg to about 40 mg of the 5α-reductase inhibitor, preferably between 4 and 10 mg, and even more preferred about 4 mg.

10. A composition according to claim 1, wherein the administration of said composition comprises administering one or two dosages of the composition thereby initiating a medication period of at least six months, and wherein about a 50% reduction in the patient's baseline testosterone level shortly after administration of the second dosage, and wherein the testosterone level in the patient returns to near baseline in less than eight weeks.

11. A composition according to claim 10, wherein the two dosages are is-separated by an interval of about 48 hours

12. A composition according to claim 10, wherein the administration comprising administering an additional dosage of the composition about six months after the prior administration.

13. A composition according to claim 1, wherein the composition further comprises at least one phosphodiesterase type 5 inhibitor, e.g. sildenafil, tadalafil, vardenafil, and avanafil.

14. A pharmaceutical formulation comprising the composition according to claim 1.

15. The composition according to claim 1 for use in treating acute urinary retention and/or nocturia.
